(19) United States
(12) Patent Application Publication
(10) Pub. No.: US 2019/0358266 A1
(43) Pub. Date: Nov. 28, 2019

(54) COMPOSITIONS OF BOTULINUM TOXIN AND PLATELET-RICH PLASMA AND THE THERAPEUTIC USE THEROF

(71) Applicant: Elite Center for Men LLC, Scottsdale, AZ (US)

(72) Inventors: Carlos A Suescun, JR., Scottsdale, AZ (US); Celisse Suescun, Scottsdale, AZ (US)

(21) Appl. No.: 15/986,976

(22) Filed: May 23, 2018

(51) Int. Cl. A61K 35/19; A61K 35/16; A61K 38/48; A61K 9/00; A61P 15/10

(52) U.S. Cl. CPC A61K 35/19; A61K 35/16; A61P 15/10; A61K 9/0019; A61K 38/4893

(57) ABSTRACT

Compositions and methods are disclosed for treating erectile dysfunction in a patient. A sample of whole blood is collected from the patient, and a sample of platelet-rich plasma (PRP) is extracted from the sample of whole blood. The sample of PRP is mixed with a predetermined amount of botulinum toxin (BTX) to form a PRP/BTX mixture, which is then injected into a region of human tissue of the patient.

### TECHNICAL FIELD

The present invention relates, generally, to compositions of botulinum toxin and platelet-rich plasma, and in particular, to the therapeutic use of such compositions in connection with, for example, the treatment of erectile dysfunction.

### BACKGROUND

Erectile dysfunction (ED), a condition characterized by the inability to achieve and/or maintain an erection of the penis during sexual activity, affects a large number of men. Since male sexual arousal involves a complex interplay of the brain, hormones, nerves, muscles, and blood vessels, erectile dysfunction can result from a deficiency in any of these systems.

Under normal conditions, stimulation of the penile shaft leads to the secretion of nitric oxide (NO), which causes the relaxation of the smooth muscles of the corpora cavernosa (the main erectile tissue of the penis), and ultimately to penile erection. More particularly, erection is initiated by external stimuli acting through somatic and autonomic pathways. Sensory receptors in the penile skin, glans, urethra, and corpus cavernosum join with the dorsal nerves of the penis and the pudendal nerve, which interfaces with the thalamus and sensory cortex. Activation of parasympathetic pathways prompts release of nitric oxide (NO) from cavernous nerves and endothelial cells, resulting in penile cavernosal smooth muscle relaxation, reducing peripheral arteriolar resistance and permitting blood inflow.

Similarly, several factors contribute to penile detumescence. Norepinephrine, phenylephrine, and endothelin activate phospholipase C, catalyzing the formation of inositol triphosphate and diacylglycerol and mediating an increase in intracytoplasmic calcium with subsequent smooth muscle contraction. Phosphodiesterase-mediated degradation of cGMP to GMP phosphodiesterase type 5 (PDE5) and cAMP to AMP (PDE4) can also prompt detumescence, hence the popular use of PDE5 inhibitors, such as VIAGRA, CIALIS, and the like to treat erectile dysfunction.

There are many known causes of ED, including, for example, diabetes mellitus, hypertension, hypogonadism, obesity, sedentary lifestyle, endocrine disorders, alcohol and drug use, depression, lower urinary tract symptoms, and cardiovascular disease. Notwithstanding the wide range of causes, substantial advances have been made toward understanding the pathophysiology of ED, leading to a variety of treatments, including oral therapies, transurethral therapies, surgical intervention via revascularization, and the deployment of prosthetic devices.

Known ED treatments are unsatisfactory in a number of respects. For example, transurethral therapies, revascularization, and prosthetics can be painful, highly invasive, and/or inconvenient. On the other hand, oral therapies, such as the various PDE5 inhibitors available as prescription drugs, can give rise to a wide range of side effects, such as headaches, flushing, upset stomach, abnormal vision, congestion, back pain, muscle pain, dizziness, and/or nausea. These side effects can make the use of such medicines by some individuals inconvenient or impossible.

Accordingly, systems and methods are needed which overcome these and other limitations of the prior art.

### SUMMARY OF THE INVENTION

In accordance with one embodiment, a method of treating a patient includes collecting a sample of whole blood from the patient, extracting a sample of platelet-rich plasma (PRP) from the sample of whole blood, mixing the sample of PRP with a predetermined amount of botulinum toxin (BTX) to form a PRP/BTX mixture, and injecting the PRP/BTX mixture into a region of human tissue of the patient.

A composition in accordance with one embodiment comprises a therapeutically effective amount of a botulinum toxin (BTX) and platelet-rich plasma (PRP), wherein the composition is suitable for injection into a human.

A method of treating erectile dysfunction in a patient in accordance with one embodiment includes injecting a therapeutically effective composition of botulinum toxin (BTX) and platelet-rich plasma (PRP) extracted from the patient into a corpora cavernosa of the patient, wherein the composition comprises the BTX in an amount between 5.0 and 40.0 LD50 units per 1.0 ml of PRP.

Various other embodiments, aspects, and features are described in greater detail below.

### BRIEF DESCRIPTION OF THE DRAWING FIGURES

The present invention will hereinafter be described in conjunction with the appended drawing figures, wherein like numerals denote like elements, and:

FIG. 1 depicts an example method for collecting whole blood from a patient;

FIG. 2 is a graphical depiction of an exemplary sample tube containing whole blood and its various constituents;

FIG. 3 is a conceptual top view of a centrifuge in accordance with an example embodiment;

FIG. 4 is a graphical depiction of an exemplary sample tube containing separated blood components;

FIG. 5 depicts the insertion of a syringe needle into the sample tube shown in FIG. 4;

FIG. 6 depicts the aspiration of platelet-poor plasma (PPP) from the sample tube shown in FIG. 5;

FIG. 7 depicts the replacement the syringe shown in FIG. 6 with a second syringe;

FIG. 8 depicts the mixing of PRP and residual PPP in the barrel of the sample tube shown in FIG. 7;

FIG. 9 depicts the aspiration of PRP into the syringe shown in FIG. 8;

FIG. 10 depicts the replacement of the needle shown in FIG. 9 with a second needle, and the provision of a vial of BTX;

FIG. 11 depicts the insertion and aspiration of BTX from the BTX vial shown in FIG. 10;

FIG. 12 depicts the hand-mixing of BTX and PRP in the barrel of the syringe shown in FIG. 12;

FIG. 13 is a partial cross-section view of the PRP/BTX mixture of FIG. 12 being injected subcutaneously into human tissue; and

FIGS. 14 and 15 depict in side and cross-sectional views, respectively, the injection of a PRP/BTX mixture into a patient's corpus cavernosum;

FIG. 16 is a flowchart illustrating an exemplary method of preparing and administering a PRP/BTX mixture;

FIG. 17 is a flowchart illustrating a method of extracting and mixing PRP and BTX in accordance with one embodiment; and

FIG. 18 is a flowchart illustrating a method of treating erectile dysfunction in accordance with one embodiment.

### DETAILED DESCRIPTION OF PREFERRED EXEMPLARY EMBODIMENTS

The following detailed description of the invention is merely exemplary in nature and is not intended to limit the invention or the application and uses of the invention. Furthermore, there is no intention to be bound by any theory presented in the preceding background or the following detailed description.

Various embodiments of the present invention relate to systems, compositions, and methods for utilizing a therapeutic mixture of platelet-rich plasma (PRP) and botulinum toxin (BTX) to treat various maladies, such as erectile dysfunction in male patients.

In accordance with one such method, a sample of whole blood is collected from the patient, and a sample of platelet-rich plasma (PRP) is extracted from the sample of whole blood (e.g., through the use of a centrifuge). The sample of PRP is mixed (e.g., in the barrel of the same syringe used to extract the PRP sample) with a predetermined amount of botulinum toxin (BTX) to form a PRP/BTX mixture, which is then injected into a region of human tissue of the patient, such as the corpora cavernosa of the patent's penis. Multiple treatments may be administered during separate sessions, and the ratio of BTX to PRP may be gradually increased until the desired clinical response has been achieved.

As described in further detail below, it has been found by the present inventors that PRP and BTX as prepared and administered herein act synergistically, and provide a non-obvious, greater than expected result when used to treat erectile dysfunction. That is, while prior art methods might contemplate using penile injection of PRP alone, or penile injection of BTX alone, none contemplate creating a mixture of the two components and injecting the mixture as described herein in a way that achieves synergistic results.

Botulinum Toxin

Botulinum neurotoxin (abbreviated herein as “BTX” or “BoNT”) is a toxin derived from a strain of the bacterium Clostridium botulinum that acts at the neuromuscular junction by inhibiting release of acetylcholine. The denervating effect of BTX results from the fact that, absent acetylcholine, the affected muscle does not receive the signal necessary to cause the muscle to contract. Although a deadly toxin at higher concentrations and quantities, various preparations of BTX have been used as a therapeutic for the treatment of neuromuscular diseases as well as sensory disorders and cosmetics.

There are eight immunotypes of BTX, designated “A” though “H”, with types “A” and “B” (i.e., BTX-A and BTX-B) being most often used for medical purposes in humans. Commercial forms of BTX include, for example, BOTOX, DYSPORT, and XEOMIN (all of which are BTX-A) and MYOBLOC (BTX-B). The term “BTX” as used herein is generally used to refer to any form of botulinum neurotoxin of a type having therapeutic value in treating human patients. In that regard, the term “BOTOX” may be used herein without loss of generality to refer to BTX-A.

The pharmaceutical measurement of the biologic activity (i.e., the denervating effect) of BTX is expressed as a “unit.” This unit is generally determined statistically by injecting cohorts of mice (specifically, an 18-22 gram Swiss-Webster mouse) at different dilutions from purified BTX protein and its protein complexes, and then determining the dilution that causes a death rate of 50% in the cohort. Thus, the pharmaceutical measurement of BTX may also be expressed an “LD50 unit,” where “LD50” is a toxicological dose descriptor meaning “lethal dose 50%”.

BTX is typically received in powder form and subsequently reconstituted using sterile saline prior to injection. For example, in some applications, BTX is reconstituted using 1.25 ml of 0.9% non-preserved sterile saline for every 50 LD50 units of BTX. In other applications, more or less sterile saline is employed as a diluent. Regardless of concentration, it is this liquid form of BTX that is then aspirated into a syringe and injected in the area of interest.

Platelet Rich Plasma

Human blood generally consists of liquid plasma and a number of solid components, such as red blood cells, white blood cells, and platelets. Platelet-rich plasma (PRP) is thus a concentrate derived from whole blood that has been processed (e.g., via one or more centrifugation and aspiration steps) to substantially remove the red blood cells and further increase the ratio of platelets to plasma.

Platelets play a role in many processes, such as coagulation, immune response, angiogenesis, and healing of damaged tissue. The alpha-granules of platelets include various proteins classified as growth factors, such as platelet-derived growth factor (PDGF), transforming growth factor (TGF), platelet factor interleukin (IL), platelet-derived angiogenesis factor (PDAF), vascular endothelial growth factor (VEGF), epidermal growth factor (EGF), insulin-like growth factor IGF, and fibronectin. Because of the high concentration of growth factors in PRP, it has been used to varying degrees in the fields of plastic surgery, orthopedics, and dermatology.

Synergistic Action of PRP and BTX

As discussed above, embodiments of the present invention relate to systems, compositions, and methods for utilizing a therapeutic mixture of platelet-rich plasma (PRP) and botulinum toxin (BTX) to treat, among other things, erectile dysfunction in male patients. It has been found by the present inventors that, while PRP and BTX work to some extent individually to address ED, these components also work synergistically.

In this regard, the ratio of components in the administered PRP/BTX composition may vary based on a number of factors, such as the age of the patient, the number of previous treatments, and the efficacy of previous treatments. In various embodiments, the composition includes BTX in an amount between 5.0 and 40.0 LD50 units per 1.0 ml of PRP. For BTX solutions in which the BTX is reconstituted using 1.25 ml of sterile saline for every 50 LD50 units of BTX, this corresponds volumetrically to using about 50-300 LD50 units of BTX for about 8.0 ml of PRP—the amount of PRP that can typically be extracted from 20-24 ml of whole blood. In such cases, the total amount of PRP/BTX injected during a session will range from between about 9.0 and 14.0 ml. It will be appreciated that the above ratios, dilutions, and volumes are merely presented as examples, and that the range of effective ratios and volumes are not so limited.

The corpus cavernosum includes a combination of elastic fibers, unstriated muscle, arteries, veins, and collagenous tunica albuginea (collagen). Accordingly, oxygen-rich blood is one of the most important components for erectile health, as oxygen helps prevent an overproduction of collagen and the build-up of collagen that can cause a decrease in blood flow. Oxygen enhances the activity of prostaglandin E1, which is produced during an erection by the smooth muscle cells in the penis. This vasodilator activates an enzyme that initiates calcium release by the smooth muscle cells, which relaxes them and allows blood flow. Prostaglandin E1 also suppresses production of collagen.

PRP's works via natural human grown factors that stimulate collagen growth and cell and nerve rejuvenation, which in turn causes the release of more nitric oxide. Nitric oxide stimulates production of cyclic GMP, a chemical that relaxes the smooth muscles in the penis. This allows blood to flow into the tiny pool-like cavernous sinuses, flooding the penis. The smooth muscles regulating the many tiny blood vessels also stay contracted, limiting the amount of blood that can collect in the penis.

BTX works through relaxation of the cavernosal smooth muscles by thwarting the release of neurotransmitter norepinephrine, which prevents blood flow but does not prevent the release of nitric oxide, which is vital to erections. Studies have shown that any therapy that can increase cavernosal smooth muscle relaxation can turn PDE51 non-responders into responders. Meaning that patients who are unable to respond to vasodilators such as VIAGRA and CIALIS are thereby able to respond.

It has been found that PRP and BTX complement each other's respective functions. While PRP rejuvenates blood vessels and nerve cells, which helps release nitric oxide, BTX relaxes the smooth muscles to allow for more oxygen-rich blood-flow while also stimulating the release of nitric oxide, resulting in complete penile rejuvenation. Trials have shown longer, firmer, and more sustainable erections, increased stamina, increase in girth and length due to new cell formation and increased blood flow, and reversal of physiological and psychological ED.

Preparation of PRP/BTX Mixture

PRP/BTX compositions as described above may be prepared in a variety of ways. In general, however, the process includes collecting a sample of whole blood from the patient, extracting a sample of PRP from the sample of whole blood, then mixing the sample of PRP with a predetermined amount of BTX (e.g., within the barrel of the syringe itself).

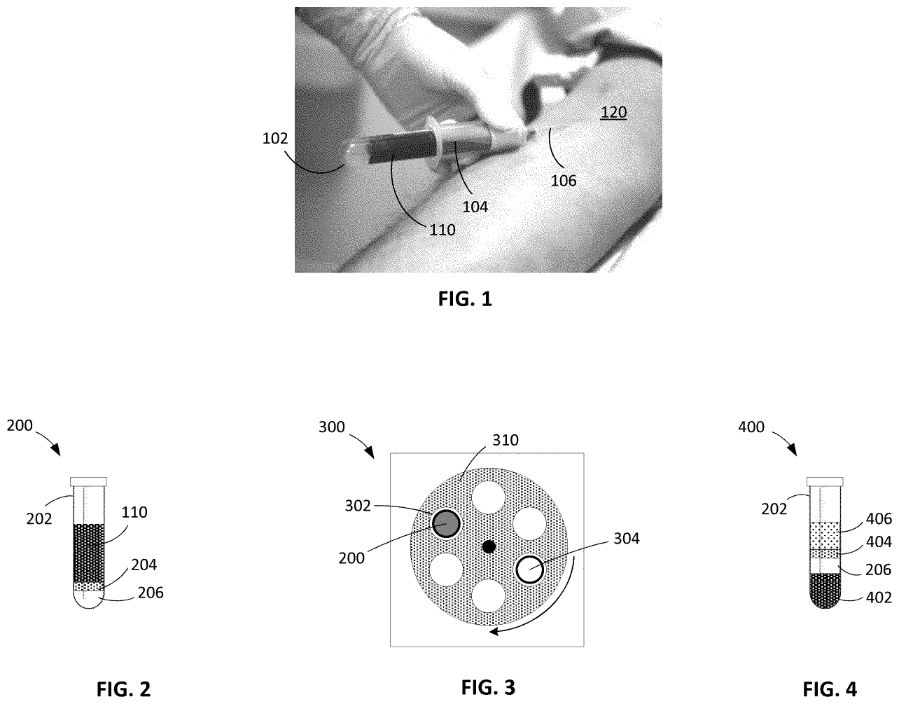

FIG. 1 illustrates an example method for drawing blood using venepuncture. More particularly, a sample of whole blood 110 is collected in a sample tube 102 via the use of a standard tube holder 104 and needle 106 inserted, for example, in the median cubital vein or other vein presented by the antecubital fossa (i.e., “elbow pit.) of a patient 120. This method of drawing blood is a standard phlebotomist technique known in the art, and need not be described in further detail herein. Any other suitable method of drawing whole blood may be used. The volume of whole blood 110 may range, for example, from 20.0 to 25.0 ml, as that volume is relatively non-invasive and is able to produce a sufficient volume of PRP for the preparation and administering of the PRP/BTX mixture.

In some embodiments, as shown in FIG. 2, the sample 200 includes a separator gel 206 and anti-coagulant 204 provided within a sample tube (or “PRP tube”) 202, which may correspond to sample tube 102 of FIG. 1. Anti-coagulant 204 prevents the coagulation of the collected whole blood 110, and separator gel 206 assists in blood component separation, as described in further detail below.

FIG. 3 illustrates, conceptually, the use of a centrifuge 300 to cause separation of the components of sample 200. As illustrated, centrifuge 300 includes a rotor 310 and a number of openings 302 for accepting samples (e.g., sample 200 as illustrated). In the interest of clarity, other typical components of centrifuge 300, such as the motor, motor controller, etc., are not illustrated. The openings 302 may be fixed angle or “swinging buckets”, as is known in the art. In the event only a single sample 200 is being centrifuged, a counterweight (e.g., a sample 304 consisting of a saline solution having a mass equal to that of sample 200) is inserted within rotor 310 in an opening that is antipodal to opening 302 of sample 200. Single-stage or multi-stage centrifugation may be employed to achieve separation. In one embodiment, for example, centrifuge 300 is operated at 3500 RPM for approximately 10 minutes. It will be understood that the time and angular speed of centrifuge 300 necessary for PRP separation will vary depending upon a wide range of factors.

Regardless of the method used for separation, the result will be substantially as shown in FIG. 4, i.e., the sample 400 will be now include, ordered from top to bottom, platelet-poor plasma (PPP) 406, PRP 404, separator gel 206, and red blood cells (and granulocytes) 402. Thus, the separator gel 206 helps create a physical border to simplify aspiration of PRP, as described in further detail below.

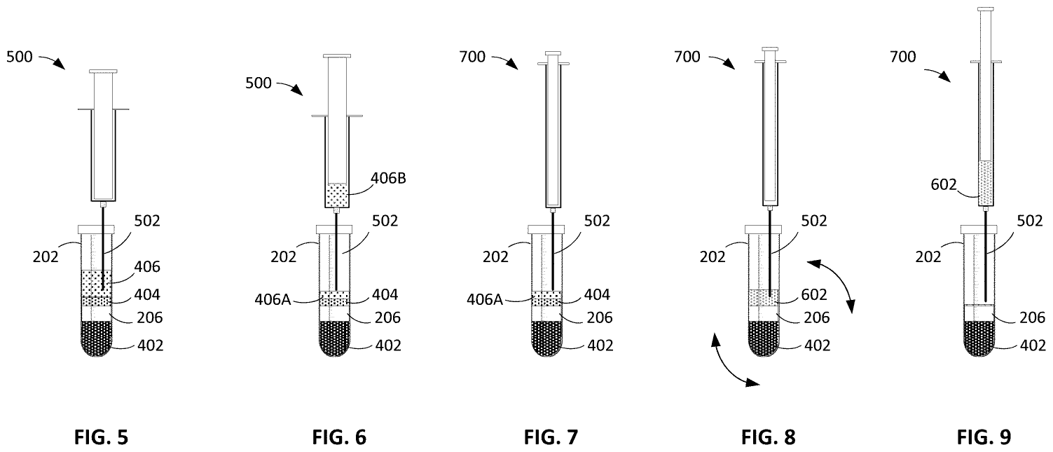

FIGS. 5-9 illustrate, sequentially, one method of extracting PRP from the separated sample 400 of FIG. 4. That is, referring first to FIG. 5, the needle 502 of syringe 500 (e.g., a “PPP syringe”) is inserted within sample tube 202 to a depth just above the bottom of PPP 406, avoiding insertion into PRP 404. This step may first require placing venting needles (not shown) to break a vacuum formed within tube 202 by a lid that seals the top of tube 202.

As is known in the art, a syringe (such as syringe 500) generally includes a barrel, a plunger (manually actuated to cause aspiration), and a tip to which a needle (such as needle 502) is removeably attached. While not separately called out in the figures, it will be understood that a hypodermic needle such as those illustrated herein generally includes a hub (which connects to the syringe tip), a shaft, and a bevel defining a tip. The hollow bore of the needle shaft is referred to as the “lumen.” Further, needle size is designated by length and gauge, where length is measured from the juncture of the hub to the tip, and gauge is a numerical designation associated with the outer diameter (with smaller gauges corresponding to larger outer diameters).

Referring now to FIG. 6, syringe 500 is used to aspirate a portion 406B of PPP into its barrel, leaving behind in tube 202 a small amount of residual PPP 406A. Next, as shown in FIG. 7, the first syringe 500 is replaced with a second syringe 700 (also referred to as a “PRP syringe”) that is more appropriate for the preparation and administration of the resulting mixture, as described below.

Next, the residual PPP 406A and PRP 404 are mixed, resulting in the configuration shown in FIG. 8. The arrows shown in FIG. 8 represent the rocking and/or other agitation of tube 202 (and connected syringe 700) to form the resulting PRP portion 602 (which will be a slightly less platelet-rich version of PRP 404). This mixing may be effected by hand, e.g., via rocking tube 200 back and forth about 10 times by hand. Referring now to FIG. 9, the PRP portion 602 is then aspirated into the barrel of syringe 700 via proper placement of needle 502.

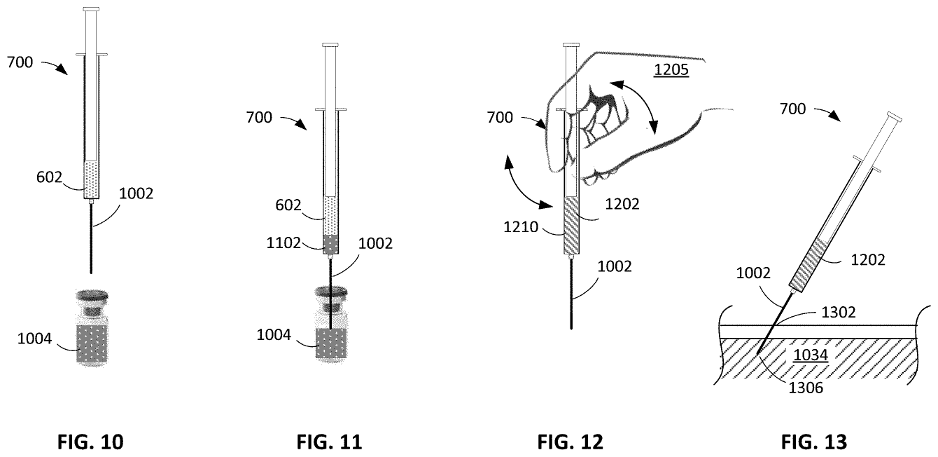

Having thus prepared and isolated the PRP portion 602, FIGS. 10-12 illustrate one method of forming the PRP/BTX mixture in accordance with one embodiment. More particularly, referring first to FIG. 10, the previous needle (needle 502 in FIG. 9) may be replaced with a second needle 1002 that is more appropriate for aspirating BTX. In one embodiment, for example, needle 1002 is a 25G, 1.5-inch needle.

Also illustrated in FIG. 10 is a BTX vial 1004 containing any suitable volume and concentration of reconstituted BTX 1004 (e.g., BTX-A, or BOTOX). In one embodiment, BTX 1004 contains a solution consisting of 1.25 ml of saline per 50 LD50 units of BTX.

Next, as illustrated in FIG. 11, needle 1002 is inserted into BTX vial 1004 and a predetermined amount of BTX (1102) is aspirated into syringe 700 along with PRP portion 602. As discussed above, the number of units (and consequently the volume) of BTX 1102 may vary. In one embodiment, in which the BTX is reconstituted at 1.25 ml per 50 units, the volume aspirated may range from 1.25 to 6 ml (corresponding to 50-300 units). In one embodiment, BTX 1102 is aspirated in an amount that results in between 5.0 and 40.0 LD50 units per 1.0 ml of PRP 602.

As shown in FIG. 12, syringe 700 is then agitated to produce the desired PRP/BTX mixture 1202. In some embodiments, syringe 700 is hand agitated (represented by hand 1205 and arcuate arrows) to form the mixture. For example, syringe 700 may be rocked back and forth manually approximately 10 times. It will be appreciated that this inter-hypodermic mixing of PRP and BTX (and, more generally, the interplay of needles and syringes illustrated in FIGS. 5-12) results in a simplified, efficient preparation method that avoids other, more time-consuming mixing methods and the potential for contamination that results from transferring liquids between vessels.

Administering the PRP/BTX Mixture

Having thus prepared the PRP/BTX mixture (e.g., mixture 1202 as shown in FIG. 12), the composition is then injected into the appropriate tissue of the patient. This is illustrated conceptually in FIG. 13, which shows needle 1002 being inserted at a site 1302 to reach a target region 1306 of tissue 1034. As described in further detail below, for the treatment of ED, tissue 1034 might correspond to one or more of the corpora cavernosa of the human penis.

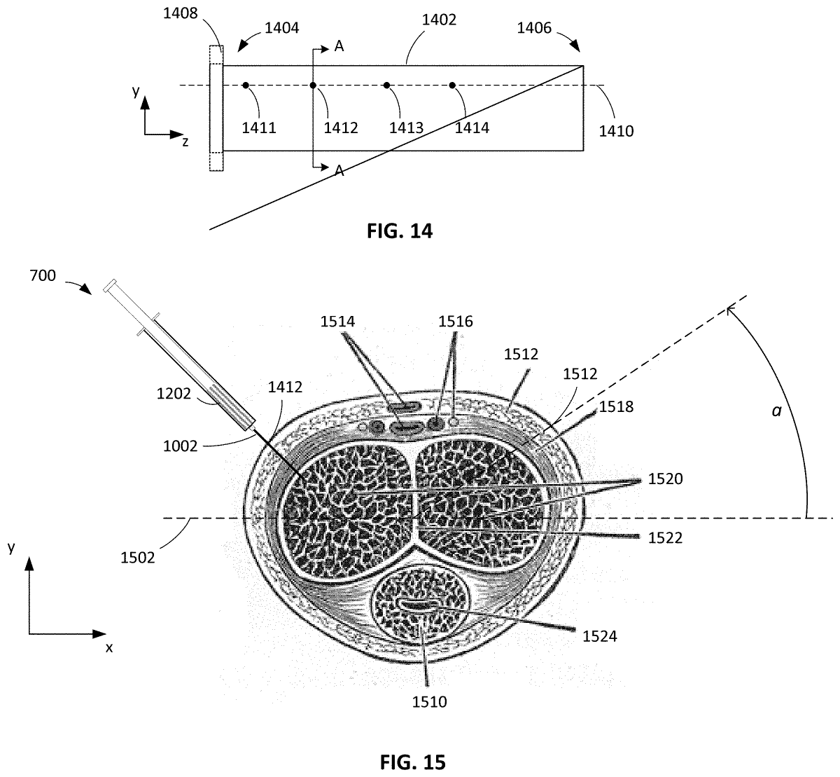

More particularly, FIGS. 14 and 15 together illustrate a human penis 1402 useful in describing methods in accordance with various embodiments. That is, FIG. 14 is a simplified side view of an exemplary penis 1402, and FIG. 15 is a cross-sectional view at section A-A of FIG. 14.

Referring to FIG. 14, penis 1402 has a proximal end 1404 (toward the body of the patient), and a distal end 1406. As shown in the cross-section of FIG. 15, penis 1402 includes two corpora cavernosa 1520 (each individually referred to herein as a corpus cavernosum) and a urethral corpus cavernosum 1510 surrounding a urethra 1510. The phrase “corpus cavernosum” and “corpora cavernosa” as used herein in connection with the injection of PRP/BTX refers to the penile cavernosa (1520), rather than the urethra cavernosum (1510).

With continued reference to FIG. 15, penis 1402 further includes dorsal artery/nerve 1516, dorsal veins 1514, integument 1512, fibrous envelope 1518, and septum pectiniforme 1522 (separating the two corpora cavernosa). Also shown in FIG. 15 is a lateral center line 1502 by which an angle (a) of injection can be described for specifying injection sites.

In accordance with one embodiment, treatment for ED proceeds as follows. First, the patient is placed on a medical procedure table (not illustrated) in supine position. Triple anesthetic cream or another topical anesthetic is applied to the shaft of penis 1402 about 10 minutes prior to the procedure. The shaft of penis 1402 is cleaned with an alcohol swab to remove the previously applied anesthetic cream.

An appropriate anesthetic is then injected at various locations into penis 1402 and/or the surrounding tissue. In one embodiment, 1% lidocaine is used for a dorsal penile block by inserting the needle into the symphysis pubis and then withdrawing the needle and reinserting it slightly inferior 2-3 mm below the previous depth. Lidocaine is then injected laterally to this landmark on both side of the midline of penis 1402, injecting about 0.5-1.0 ml of lidocaine with each injection.

Approximately 5.0 to 10.0 minutes after the dorsal penile block takes effect, an elastic ring (1408 in FIG. 14) is placed at the base of penis 1402 to allow for the PRP/BTX mixture to act locally.

Two to five (in some embodiments, three to four) sites along the corpus cavernosum 1520 are then injected with approximately 0.5 to 1.0 ml of the PRP/BTX mixture so that substantially equal volumes are injected at each site. This is illustrated in FIG. 14, which shows four injection sites 1411-1414 along a longitudinal line 1410 on one side of penis 1402, and needle 1002 entering an injection site 1412 in FIG. 15. Prior to injection, the needle previously used (e.g., as shown in FIGS. 10-12) may be replaced with a needle more appropriate for injection, such as a 30G, 0.5 inch needle. This size needle has been found effective in reaching the corpora cavernosa 1520 through the integument 1512 and fibrous envelope 1518.

In some embodiments, the injections are made at the “10 o'clock” and “2 o'clock” positions of the penis, corresponding roughly to angles a of 45 degrees and 135 degrees in FIG. 15 (and shown as line 1410 in FIG. 14).

The elastic ring 1408 is maintained in place for approximately 30 minutes after completion of all injection sites to allow the PRP/BTX mixture to act locally along corpora cavernosa 1520. Subsequently, elastic ring 1408 is removed and the patient may resume his normal daily activities.

The above treatment may be repeated, for example, every 10 to 30 days. The ratio of BTX to PRP in the mixture, the number and placement of injection sites, and the frequency of treatments may all be modulated over time to achieve a particular clinical result. These results may be measured using a variety of qualitative and quantitative metrics known in the art. Such metrics include, for example, an erectile hardness grading scale (EHS), nocturnal penile tumescence activity, psychometric quality-of-life (QoL) testing, questionnaires (such as the Sexual Health Inventory for Men, or SHIM), and the like.

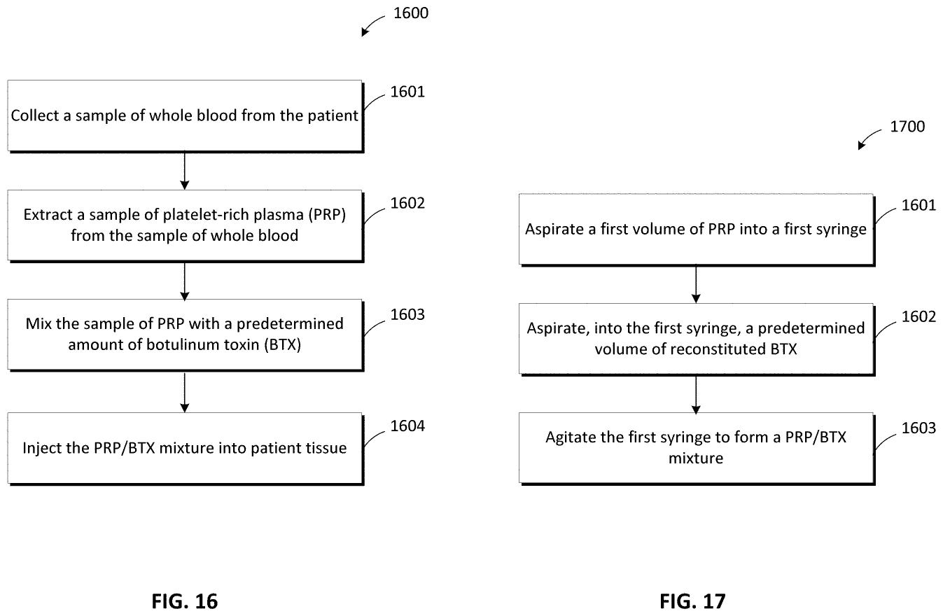

FIGS. 16-18 are flowcharts that outline, at a high level, the methods described in greater detail above. That is, FIG. 16 depicts a method 1600 including collecting a sample of whole blood from the patient (1601), extracting a sample of PRP from the sample of whole blood (1603), and injecting the PRP/BTX mixture into human tissue (1604). FIG. 17 illustrates one sub-method 1700 for accomplishing the mixing step 1603. More particularly, the method includes aspirating a first volume of PRP into a first syringe (1601), aspirating, into the first syringe, a predetermined volume of the reconstituted BTX (1602), and agitating the first syringe to form a PRP/BTX mixture (1603). Thus, method 1700 illustrates the inter-syringe mixing of PRP and BTX to form the desired mixture in the same syringe (i.e., same syringe barrel) that was used to aspirate the PRP.

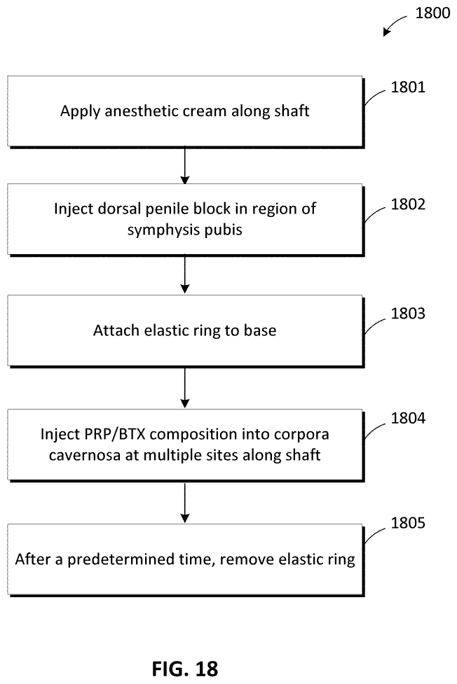

FIG. 18 depicts, at a high level, an exemplary method 1800 for penile injection of PRP/BTX as more fully discussed above. Specifically, method 1800 includes applying an anesthetic cream along the shaft of the penis (1801), injecting a dorsal penile block (e.g., in the symphysis pubis region) (1802), attaching an elastic ring to the base of the penis (1803), injecting a PRP/BTX mixture into the corpora cavernosa of the penis at multiple sites along its shaft (1804), and, after a predetermined length of time (e.g., 30 minutes), removing the elastic ring (1805).

In summary, what has been disclosed are systems, methods, and compositions for treating patients (e.g., for erectile dysfunction) using a mixture of BTX and PRP.

In one embodiment, a composition comprises a therapeutically effective amount of a botulinum toxin (BTX) and platelet-rich plasma (PRP), wherein the composition is suitable for injection into a human. In some embodiments, the BTX is present in an amount between 5.0 and 40.0 LD50 units per 1.0 ml of PRP. In some embodiments, the botulinum toxin is immunotype A (BTX-A). The composition may be suitable for injection into a corpus cavernosum of a human male and is therapeutically effective for treating erectile dysfunction.

In accordance with one embodiment, a method of treating erectile dysfunction in a patient includes injecting a therapeutically effective composition of botulinum toxin (BTX) and platelet-rich plasma (PRP) extracted from the patient into a corpora cavernosa of the patient, wherein the composition comprises BTX in an amount between 5.0 and 40.0 LD50 units per 1.0 ml of PRP. In one embodiment, the method includes injecting the therapeutically effective composition of BTX and PRP at a rate of one injection every 20-40 days.

The composition of BTX and PRP may be a mixture produced by agitating a syringe barrel containing the BTX and the PRP. The PRP/BTX mixture may be injected into the corpus cavernosum at a plurality of sites such that a substantially equal amount of the PRP/BTX mixture is injected at each of the plurality of sites. The total volume of the PRP/BTX mixture injected into the corpus cavernosum is between 9 and 14 ml. The patient may receive the treatment for erectile dysfunction on multiple days, such that the predetermined amount of BTX is progressively increased during the plurality of days until a desired clinical response is achieved.

In accordance with one embodiment, a method of treating erectile dysfunction in a patient includes collecting a sample of whole blood from the patient; extracting a sample of platelet-rich plasma (PRP) from the sample of whole blood; mixing the sample of PRP with a predetermined amount of botulinum toxin (BTX) to form a PRP/BTX mixture; and injecting the PRP/BTX mixture into a region of human tissue of the patient. The predetermined amount of BTX may be selected such that the BTX is present in an amount between 5.0 and 40.0 LD50 units per 1.0 ml of PRP.

In one embodiment, mixing the sample of PRP with the predetermined amount of BTX includes aspirating the predetermined amount of BTX into a first syringe used to extract the sample of PRP, and agitating the first syringe to form the PRP/BTX mixture. In one embodiment, injecting the PRP/BTX mixture includes injecting the PRP/BTX mixture into a corpus cavernosum of the penis of the patient. In one embodiment, the method further includes placing an elastic ring about the base of the penis prior to injecting the PRP/BTX mixture. In one embodiment, the PRP/BTX mixture is injected with a 30G needle having a length of about 0.5 inches. The PRP/BTX mixture may be injected into the corpus cavernosum at multiple sites such that a substantially equal amount of the PRP/BTX mixture is injected at each site.

In one embodiment, the total volume of the PRP/BTX mixture injected into the corpus cavernosum is between 9 and 14 ml. The patient may receive the treatment for erectile dysfunction on a plurality of days, and the predetermined amount of BTX is progressively increased during the plurality of days until a desired clinical response is achieved. In some embodiments, the plurality of days occur regularly at approximately once per month.

While much of the foregoing applications of PRP/BTX are discussed in the context of treating erectile dysfunction, the invention is not so limited, and may be utilized in a variety of other contexts and to treat a number of other maladies in both male and female patients. For example, the mixture may be injected other corpora cavernosa tissue present within the human body. Indeed, compositions and methods in accordance with the present invention may in connection with the treatment of mammals other than human beings.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

1. A composition comprising a therapeutically effective amount of a botulinum toxin (BTX) and platelet-rich plasma (PRP), wherein the composition is suitable for injection into a human.

2. The composition of claim 1, wherein the BTX is present in an amount between 5.0 and 40.0 LD50 units per 1.0 ml of PRP.

3. The composition of claim 1, wherein the botulinum toxin is immunotype A (BTX-A).

4. The composition of claim 1, wherein the composition is suitable for injection into a corpus cavernosum of a human male and is therapeutically effective for treating erectile dysfunction.

5. A method of treating erectile dysfunction in a patient, the method comprising injecting a therapeutically effective composition of botulinum toxin (BTX) and platelet-rich plasma (PRP) extracted from the patient into a corpora cavernosa of the patient, wherein the composition comprises BTX in an amount between 5.0 and 40.0 LD50 units per 1.0 ml of PRP.

6. The method of claim 5, wherein method includes injecting the therapeutically effective composition of BTX and PRP at a rate of one injection every 20-40 days.

7. The method of claim 5, wherein the composition of BTX and PRP is a mixture produced by agitating a syringe barrel containing the BTX and the PRP.

8. The method of claim 5, wherein the PRP/BTX mixture is injected into the corpus cavernosum at a plurality of sites such that a substantially equal amount of the PRP/BTX mixture is injected at each of the plurality of sites.

9. The method of claim 8, wherein the total volume of the PRP/BTX mixture injected into the corpus cavernosum is between 9 and 14 ml.

10. The method of claim 5, further comprising successively injecting the patient with the PRP/BTX mixture on multiple days, while progressively increasing the predetermined amount of BTX until a desired clinical response is achieved.

11. A method of treating erectile dysfunction in a patient, the method comprising:
collecting a sample of whole blood from the patient;
extracting a sample of platelet-rich plasma (PRP) from the sample of whole blood;
mixing the sample of PRP with a predetermined amount of botulinum toxin (BTX) to form a PRP/BTX mixture; and
injecting the PRP/BTX mixture into a region of human tissue of the patient.

12. The method of claim 11, wherein the predetermined amount of BTX is selected such that the BTX is present in an amount between 5.0 and 40.0 LD50 units per 1.0 ml of PRP.

13. The method of claim 11, wherein mixing the sample of PRP with the predetermined amount of BTX includes:
aspirating the predetermined amount of BTX into a first syringe previously used to extract the sample of PRP; and
manually agitating the first syringe to mix the BTX with the PRP to form the PRP/BTX mixture.

14. The method of claim 11, wherein injecting the PRP/BTX mixture includes injecting the PRP/BTX mixture into a corpus cavernosum of the penis of the patient.

15. The method of claim 14, further including placing an elastic ring about the base of the penis prior to injecting the PRP/BTX mixture.

16. The method of claim 14, wherein the PRP/BTX mixture is injected with a 30-gauge needle having a length of about 0.5 inches.

17. The method of claim 14, wherein the PRP/BTX mixture is injected into the corpus cavernosum at multiple sites such that a substantially equal amount of the PRP/BTX mixture is injected at each site.

18. The method of claim 17, wherein the total volume of the PRP/BTX mixture injected into the corpus cavernosum is between 9 and 14 ml.

19. The method of claim 11, wherein the patient receives the treatment for erectile dysfunction on a plurality of days, and the predetermined amount of BTX is progressively increased during the plurality of days until a desired clinical response is achieved.

20. The method of claim 19, wherein the plurality of days occur regularly at approximately once per month.
